(19) United States
(12) Patent Application Publication
(10) Pub. No.: US 2011/0236275 A1
(43) Pub. Date: Sep. 29, 2011

(54) STEAMING AND SANITIZING ACCESSORY

(76) Inventors: Terry ROBERTSON, Dadeville, AL (US); Pierantonio Milanese, Susegana (IT); Andrea Milanese, Conegliano (IT)

(21) Appl. No.: 13/072,699

(22) Filed: Mar. 26, 2011

Related U.S. Application Data
(60) Provisional application No. 61/318,287, filed on Mar. 27, 2010.

(51) Int. Cl. A61L 2/07

(52) U.S. Cl. 422/291; 422/292

(57) ABSTRACT

An accessory for a steam generating appliance includes a valve having one or more inlets for receiving one or both of steam and a sanitizing agent from the steam generating appliance. One or more forwardly-directed outlet ports and one or more downwardly-directed outlet ports are included. A first conduit provides fluid communication between the valve and the one or more forwardly-directed outlet ports. A second conduit provides fluid communication between the valve and the one or more downwardly-directed outlet ports. A valve controller selectively allows or denies the one or both of steam and a sanitizing agent to flow through the valve and conduits to the outlet ports in at least several modes.

### RELATED APPLICATIONS

This Application is a formalization of and claims priority to U.S. Provisional Application No. 61/318287, filed Mar. 27, 2010, which claims priority to U.S. application Ser. No. 12/565243, filed Sep. 23, 2009, and Ser. No. 11/885994, filed Sep. 10, 2007, now U.S. Pat. No. 7,891,047, the entire teachings of which are all incorporated herein by reference. This Application also claims priority to U.S. application Ser. No 13/013956, filed Jan. 26, 2011, which claims priority to U.S. application Ser. No. 11/885994, filed Sep. 10, 2007, now U.S. Pat. No. 7,891,047, and to application Ser. No. 29/378185, filed Jan. 11, 2010, the entire teachings of which are all incorporated herein by reference.

### FIELD OF THE INVENTION

The present invention is related to steam-cleaning and disinfecting. More specifically, the invention is related to the combination of a sanitizing agent and super-heated water by an accessory for a steaming apparatus to enhance the sanitizing properties of the vapor dispensed there-from, and to an accessory to a steaming apparatus adapted therefore.

### BACKGROUND

Steam-cleaning appliances and steam sanitizing methods of an infinite variety are well known and commonly used for cleaning and attempting to sanitize floors and surfaces. Such apparatuses universally claim to sanitize and disinfect solely by directing super-heated water, also known as steam, at the object to be sanitized. A disadvantage common to such prior art is a failure to fully sanitize. It is found that such sanitizing efforts are insufficient and typically leave living microbes on the floors surfaces, which soon regenerate. It is found the steaming of the floor or surface oftentimes improves the conditions for such regeneration, eventually resulting in the floor or surface being less sanitary than had it never been exposed to the steaming in the first place.

There exists a need for improvement in the sanitizing of floors and surfaces, and such is an object of the present invention. There exists the need for improvement of steaming and steam-cleaning apparatuses, and such is an object of the present invention. There exists the need for elimination of the microbial regeneration conditions on floors and surfaces after steaming, and such as an object of the present invention. Further needs and objects exist, which are addressed by the present invention, as may become apparent by the included disclosure of an exemplary embodiment thereof.

### SUMMARY OF THE INVENTION

The invention lies in a method and/or apparatus for steaming of a floor or other surface and/or for combining steaming of a floor or other surface with the application of a sanitizing agent thereon, to more completely sanitize the floor or surface and to discourage the subsequent regeneration of microbial life.

In one exemplary embodiment, the invention may be practiced in a device for cleaning a floor or surface including a steam-cleaning apparatus and an accessory therefore, in combination; the accessory having downwardly-directed steaming ports and forwardly-directed steaming ports. A control valve allows independent dispersal of steam from only one of the downwardly-directed or the forwardly-directed ports at a time, or allows combined dispersal from both sets of ports simultaneously.

In another exemplary embodiment, the invention may be practiced in a method of sanitizing and cleaning a floor or surface using a steam-cleaning apparatus and an accessory therefore, in combination, or the invention maybe embodied in such an apparatus and accessory, in combination.

The apparatus may be of the type having first and second reservoirs, one reservoir for holding water to be vaporized into steam and the other reservoir for holding a sanitizing agent. And the accessory may have downwardly-directed steaming ports and forwardly-directed steaming ports. A control valve allows dispersal of the steam from the downwardly-directed ports or dispersal of the sanitizing agent from the forwardly-directed ports, either one at a time, or from both sets of ports simultaneously.

The method may include the steps of receiving water into the first reservoir, receiving the sanitizing agent into the second reservoir, heating the water within the first reservoir, cleaning the floor or surface by directing the heated water there-at through the accessory, dispensing the sanitizing agent onto the floor or surface via the accessory, and sanitizing the floor or surface by combining the heated water and sanitizing agent there-on via the accessory.

The step of heating the water may include heating the water to at least approximately one hundred and eighty degrees F. The step of combining the sanitizing agent and the heated water may include mixing at a volume rate of four parts sanitizing agent to ten parts water.

The sanitizing agent may include Alkyl Dimethyl Ethylbenzyl Ammonium Chloride, BTC 2125 and Chlorine Dioxide. The percentage by volume in the sanitizing agent of the Alkyl Dimethyl Ethylbenzyl Ammonium Chloride may be approximately 0.125. The percentage by volume in the sanitizing agent of the BTC 2125 may be approximately 0.125. The percentage by volume in the sanitizing agent of the Chlorine Dioxide may be approximately 0.20.

The invention may also be embodied in a method of sanitizing a floor or surface using a steaming apparatus and, in combination therewith, an accessory therefore, the method including the steps of receiving water into a reservoir of the steaming apparatus, receiving the sanitizing agent into the reservoir, heating the water and sanitizing agent, and sanitizing the floor or surface by directing the combined heated water and sanitizing agent there-at via the accessory.

The step of heating the water and sanitizing agent in this embodiment may also include heating to at least approximately one hundred and eighty degrees F. The water and sanitizing agent received into the reservoir may be received at a volume rate of approximately four parts sanitizing agent to ten parts water.

The invention may also be embodied in a steam-cleaning apparatus and, in combination therewith, an accessory therefore, for sanitizing and cleaning a floor or surface and including a first reservoir for receiving water, a heater for heating the water within the first reservoir, a second reservoir for receiving a sanitizing agent, a first actuator for enabling the heated water from the first reservoir through the accessory at the floor or surface for cleaning the floor or surface, and a second actuator for enabling the sanitizing agent from the second reservoir through the accessory at the floor or surface and for enabling the mixed heated water and sanitizing agent at the floor or surface for sanitizing the floor or surface. The heater may be adapted to heat the water to at least approximately one hundred and eighty degrees F. The accessory may be adapted to mix the sanitizing agent with the heated water at a volume rate of four parts sanitizing agent to ten parts water.

The invention may also be embodied in a steaming apparatus and, in combination therewith, an accessory therefore, for sanitizing a floor or surface and including a reservoir for receiving water and a sanitizing agent, a heater for heating the water and sanitizing agent within the reservoir, an actuator for enabling the heated water and sanitizing agent from the reservoir through the accessory at the floor or surface for sanitizing the floor or surface. The heater may be adapted to heat the water and sanitizing agent to at least approximately one hundred and eighty degrees F.

The invention may also be embodied in an accessory for a steam generating appliance having a valve having one or more inlets for receiving one or both of steam and a sanitizing agent from the steam generating appliance. The accessory may have one or more forwardly-directed outlet ports and one or more downwardly-directed outlet ports, a first conduit providing fluid communication between the valve and the one or more forwardly-directed outlet ports, and a second conduit providing fluid communication between the valve and the one or more downwardly-directed outlet ports. The accessory may have a valve controller for selectively allowing or denying the one or both of steam and a sanitizing agent to flow through the valve and conduits to the outlet ports in at least the following modes; denying the one or both of steam and a sanitizing agent to flow to and from either of the forwardly-directed or the downwardly-directed outlet ports, allowing the one or both of steam and a sanitizing agent to flow to and from both of the forwardly-directed and the downwardly-directed outlet ports, allowing the one or both of steam and a sanitizing agent to flow to and from only the one or more forwardly-directed outlet ports, and allowing the one or both of steam and a sanitizing agent to flow to and from only the one or more downwardly-directed outlet ports.

The accessory may further have a handle, a rigid tubular wand, and a dispensing head, wherein the valve is disposed in the handle, the first and second conduits are disposed in the wand, and the outlet ports are disposed in the dispensing head. The one or more downwardly-directed ports may be disposed in a bottom surface of the dispensing head, and the bottom surface may include means for removably receiving an absorbent pad. The means for removably receiving the absorbent pad may be “hook and loop” hooks.

The dispensing head may include the received absorbent pad, and the pad may include a super-absorbent material. The super-absorbent material may include hydrophilic fibers and hydrogel-forming particles. The super-absorbent material may include cotton and sodium polyacrylate.

The invention may also be embodied in an accessory for a steam generating appliance having a valve having an inlet for receiving steam from the steam generating appliance. This accessory may include one or more forwardly-directed outlet ports and one or more downwardly-directed outlet ports. This accessory may further include a first conduit providing fluid communication between the valve and the one or more forwardly-directed outlet ports, and a second conduit providing fluid communication between the valve and the one or more downwardly-directed outlet ports. This accessory may have a valve controller for selectively allowing or denying the steam to flow through the valve and conduits to the outlet ports in at least the following modes; denying the steam to flow to and from either of the forwardly-directed or the downwardly-directed outlet ports, allowing the steam to flow to and from both of the forwardly-directed and the downwardly-directed outlet ports, allowing the steam to flow to and from only the one or more forwardly-directed outlet ports, and allowing the steam to flow to and from only the one or more downwardly-directed outlet ports.

This accessory may further have a dispensing portion and an absorbent pad, wherein the one or more downwardly-directed ports are disposed in a bottom surface of the dispensing portion. The bottom surface may include means for removably receiving the absorbent pad. This absorbent pad may include an opening there-through shaped and disposed to avoid the one or more downwardly-directed ports, such that steam flowing there-from does not impact the pad.

This accessory may have a handle, a rigid tubular wand, and a dispensing portion, wherein the valve is disposed in the handle, the first and second conduits are disposed in the wand, and the outlet ports are disposed in the dispensing portion. The one or more downwardly-directed ports may be disposed in a bottom surface of the dispensing portion, and the bottom surface may include means for removably receiving an absorbent pad.

The invention may also be embodied in an accessory for a steam generating appliance having a valve having a first inlet for receiving steam from the steam generating appliance and a second inlet for receiving a sanitizing agent. This accessory may include one or more forwardly-directed outlet ports and one or more downwardly-directed outlet ports. This accessory may further include a first conduit providing fluid communication between the valve and the one or more forwardly-directed outlet ports, and a second conduit providing fluid communication between the valve and the one or more downwardly-directed outlet ports. This accessory may have a valve controller for selectively allowing or denying the steam and the sanitizing agent to flow through the valve and conduits to the outlet ports in at least the following modes; denying both the steam and the sanitizing agent to flow to and from either of the forwardly-directed or the downwardly-directed outlet ports, allowing the steam to flow to and from the one or more forwardly-directed outlet ports and the sanitizing agent to flow to and from the one or more downwardly-directed outlet ports, allowing the sanitizing agent to flow to and from the one or more forwardly-directed outlet ports and the steam to flow to and from the one or more downwardly-directed outlet ports, and allowing a mixture of the steam and the sanitizing agent to flow to and from both of the forwardly-directed and the downwardly-directed outlet ports.

The sanitizing agent may include Alkyl Dimethyl Ethylbenzyl Ammonium Chloride, wherein the percentage of Alkyl Dimethyl Ethylbenzyl Ammonium Chloride by volume of the sanitizing agent may be approximately 0.125. The sanitizing agent may include BTC 2125, wherein the percentage of BTC 2125 by volume of the sanitizing agent may be approximately 0.125. The sanitizing agent may include Chlorine Dioxide, wherein the percentage of Chlorine Dioxide by volume of the sanitizing agent may be approximately 0.20. The sanitizing agent may include Alkyl Dimethyl Ethylbenzyl Ammonium Chloride, BTC 2125, and Chlorine Dioxide, wherein the percentage of Alkyl Dimethyl Ethylbenzyl Ammonium Chloride by volume of the sanitizing agent may be approximately 0.125.

This accessory may further have a handle, a rigid tubular wand, and a dispensing head, wherein the valve is disposed in the handle, the first and second conduits are disposed in the wand, and the outlet ports are disposed in the dispensing head.

The one or more downwardly-directed ports may be disposed in a bottom surface of the dispensing head, and the bottom surface may include means for removably receiving an absorbent pad.

Further features and aspects of the invention are disclosed with more specificity in the Detailed Description, Drawings, and Appendices provided herein and showing exemplary embodiments of the invention.

### BRIEF DESCRIPTION OF THE DRAWINGS AND APPENDICES

Many aspects of the invention can be better understood with reference to the included drawings. The components in the drawings are not necessarily to scale, and all components may not be present in all views, emphasis instead being placed upon clearly illustrating the principles of the present invention. Moreover, in the drawings, like reference numerals designate corresponding parts throughout the several views.

FIG. 1 is a perspective view of a first exemplary steaming apparatus and floor steaming accessory therefore for use in practicing the invention;

FIG. 2 is a downwardly-looking perspective view of the head portion of the accessory of FIG. 1;

FIG. 3 is a downwardly-looking perspective view of the head portion of FIG. 2 having its covers removed;

FIG. 4 is a perspective view of the underside of the head portion of FIG. 2

FIG. 5 is a cross-sectional view through the accessory of FIG. 1;

FIG. 6 is a partial cross-sectional view through the accessory of FIG. 1 showing steam being dispersed from the forwardly-directed ports;

FIG. 7 is a partial cross-sectional view through the accessory of FIG. 1 showing steam being dispersed from the downwardly-directed ports;

FIG. 8 is a partial cross-sectional view through the accessory of FIG. 1 showing steam being dispersed from both sets of ports;

FIG. 9 is a partial cross-sectional view through the control valve of the accessory of FIG. 1 showing steam being allowed to the tube feeding the forwardly-directed ports;

FIG. 10 is a partial cross-sectional view through the control valve of the accessory of FIG. 1 showing steam being allowed to the tube feeding the downwardly-directed ports;

FIG. 11 is a partial cross-sectional view through the control valve of the accessory of FIG. 1 showing steam being allowed to both of the tubes;

FIG. 12 is a perspective view of a second exemplary steam-cleaning apparatus and floor cleaning/sanitizing accessory therefore for use in practicing the invention;

FIG. 13 is a downwardly-looking perspective view of the head portion of the accessory of FIG. 12;

FIG. 14 is a downwardly-looking perspective view of the head portion of FIG. 13 having its covers removed;

FIG. 15 is a perspective view of the underside of the head portion of FIG. 13;

FIG. 16 is a cross-sectional view through the accessory of FIG. 12;

FIG. 17 is a partial cross-sectional view through the accessory of FIG. 12 showing sanitizing agent being dispersed from the forwardly-directed ports;

FIG. 18 is a partial cross-sectional view through the accessory of FIG. 12 showing steam being dispersed from the downwardly-directed ports;

FIG. 19 is a partial cross-sectional view through the accessory of FIG. 12 showing sanitizing agent and steam being dispersed from both sets of ports; and

FIG. 20 is a partial cross-sectional view through the control valve of the accessory of FIG. 12 showing sanitizing agent being allowed to the tube feeding the forwardly-directed ports;

FIG. 21 is a partial cross-sectional view through the control valve of the accessory of FIG. 12 showing steam being allowed to the tube feeding the downwardly-directed ports;

FIG. 22 is a partial cross-sectional view through the control valve of the accessory of FIG. 12 showing sanitizing agent and steam being allowed to both of the tubes;

FIGS. 23 to 25 show a microbiology study report relevant to the embodiment of FIG. 12.

### DETAILED DESCRIPTION

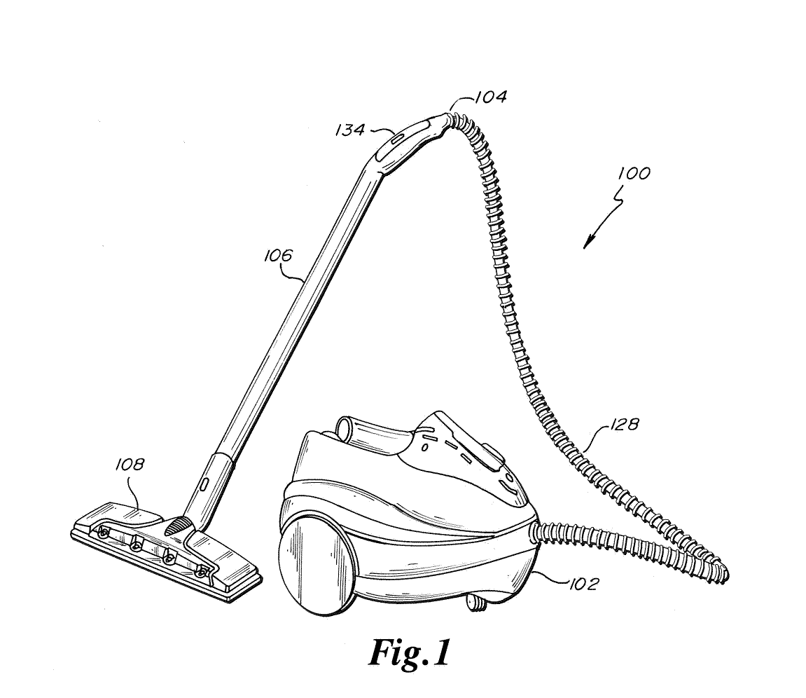

Reference is now made to FIGS. 1 through 11, where there is shown a steam cleaner 100 in the form of a canister steam generator 102 for heating water and having a reservoir for receiving the water to be heated into steam and a flexible hose 128 leading from the reservoir and canister to provide the steam. A floor cleaning accessory 104 includes a rigid hose assembly 106 and a steam-dispending head 108 connectable to the steam generator.

It should be understood that the disclosure constitutes merely a first exemplary one of the infinite number of possible embodiments for use in practicing the present invention. For instance, the steam generator 102 depicted is merely exemplary of typical such steam generators of the art and it should be understood that the invention may be practiced using another or most equivalent steam generators having means to accept water, heat the water to steam for supplying there-from, and having means to supply such steam to a dispensing means.

Water is added into an internal boiling chamber of the generator through an opening after its lid is temporarily removed. The lid is replaced after the chamber is filled, and a heating element communicating with the chamber is energized to heat the water within the chamber into steam, which is retained therein under pressure.

The rigid hose assembly 106 includes two parallel tubes 110F and 110B contained within a common rigid tubular housing 112, and a control valve 114. The control valve is actuated by a trigger 134 to force the valve into four distinct functions; “Off”, “Frontal Steam Only”, “Bottom Steam Only”, and “Frontal/Bottom Steam”. Those functions will be further explained.

The rigid hose assembly affixes in fluid communication to the flexible hose of the steam generator at the rigid hose's proximal end 130 such that the flexible hose's inlet tube 120 connects to valve 114, and the rigid hose assembly is integrated with and in fluid communication with the steam-dispensing head 108 at the hose's distal end 132. The hose assembly thereby serves as a steam conduit from the steam generator 102 to the steam-dispensing head 108.

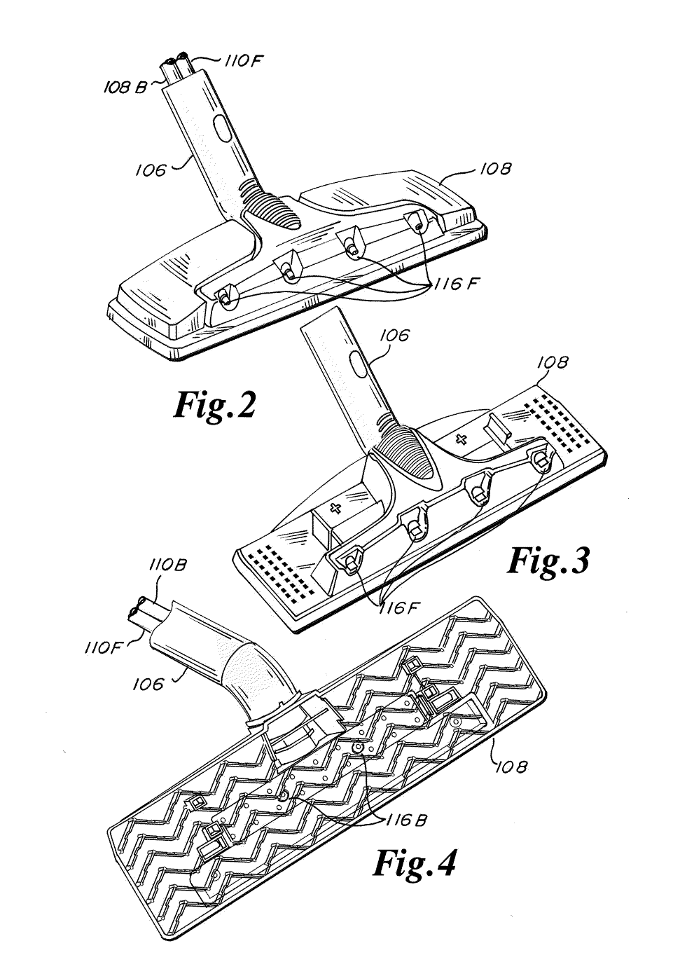

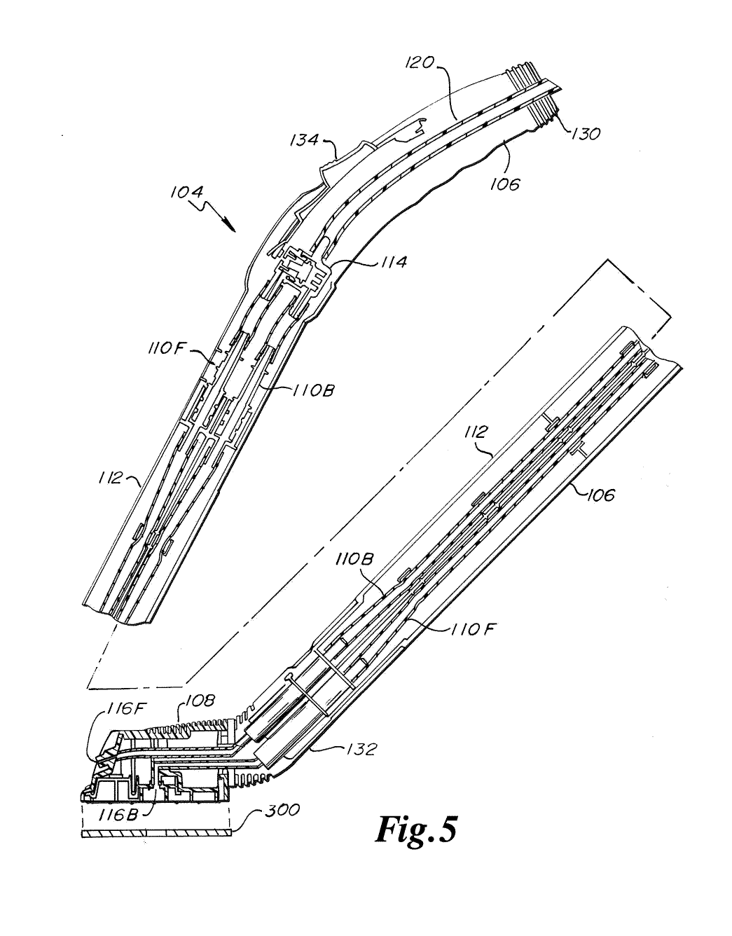

FIGS. 2 through 4 show that the dispensing head 108 is equipped with both frontal steam dispensing ports 116F, which are frontally-directed from the head, and bottom steam dispensing ports 116B, which are downwardly-directed from the head. FIG. 5 is a cross-section through the accessory 104 to show the fluid pathways there-through. The boiling chamber of the generator communicates with one or both of two tubes 110F and 110B of the hose assembly through inlet tube 120 and through the control valve 114, such that the valve normally denies escape of steam from the boiling chamber, and activation of the valve into one of three positions allows the escape of steam from the chamber to and through the rigid hose to the steam-dispensing head. Preferably, the underside of the head has removably and replaceably attachable thereto pad 300, such as, but not limited to the type disclosed in priority Application No 13/013956. Preferably, the means for removably and replaceably attaching the pad to the underside of the head are “hook and loop” hooks on the underside.

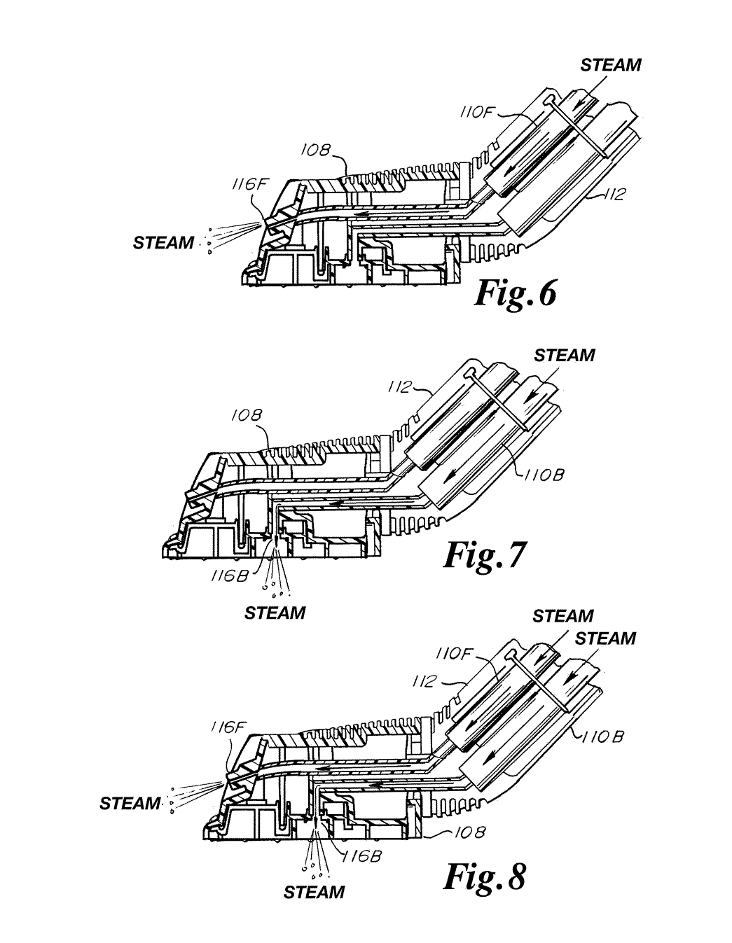

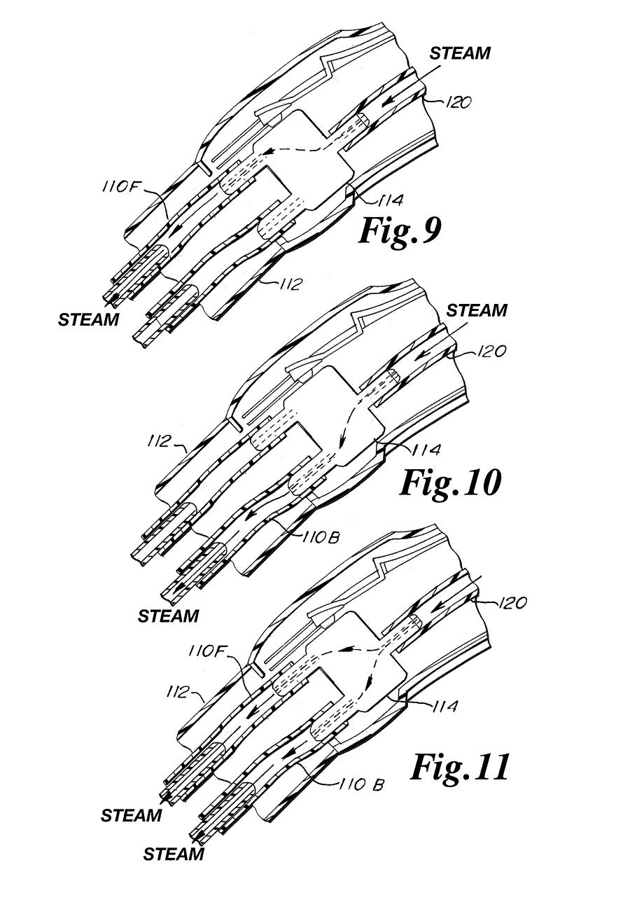

We refer now to FIGS. 6 through 8 and respectively corresponding FIGS. 9 through 11. These figures show the “Frontal Steam Only”, “Bottom Steam Only”, and “Frontal/Bottom Steam” functions, respectively. The “Off” function is not shown, as in that position valve 114 is closed and denies steam to the dispensing head completely.

Steam dispensing ahead of the dispensing head in the “Frontal Steam Only” function, such as to pre-soften residue on the floor ahead of wiping it, is accomplished by the arrangement shown in FIGS. 6 and 9. With dispensing head 108 placed upon and against the floor or surface and the trigger moved to force the control valve into the “Frontal Steam Only” position shown in FIG. 9, pressurized steam from the generator is allowed to flow through the valve to tube 110F and eventually and forcefully out through frontal ports 116F onto the floor or surface in front of the head as it is moved about the floor or surface. Wiping pad 300 will then wipe up and absorb the softened residue and the steam that condenses back to water as it contacts the floor.

Steam dispensing below the dispensing head in the “Bottom Steam Only” function, such as to penetrate and sterilize the floor as it is being wiped, is accomplished by the arrangement shown in FIGS. 7 and 10. With dispensing head 108 placed upon and against the floor or surface and the trigger moved to force the control valve into the “Bottom Steam Only” position shown in FIG. 10, pressurized steam from the generator is allowed to flow through the valve to tube 110B and eventually and forcefully out through bottom ports 116B onto the floor or surface below the head as it is moved about the floor or surface. Wiping pad 300 will then wipe up and absorb the steam that condenses back to water as it contacts the floor.

Steam dispensing both ahead of and below the dispensing head in the “Frontal/Bottom Steam” function is accomplished by the arrangement shown in FIGS. 8 and 11. In this way, both of the aforesaid functions may be accomplished simultaneously.

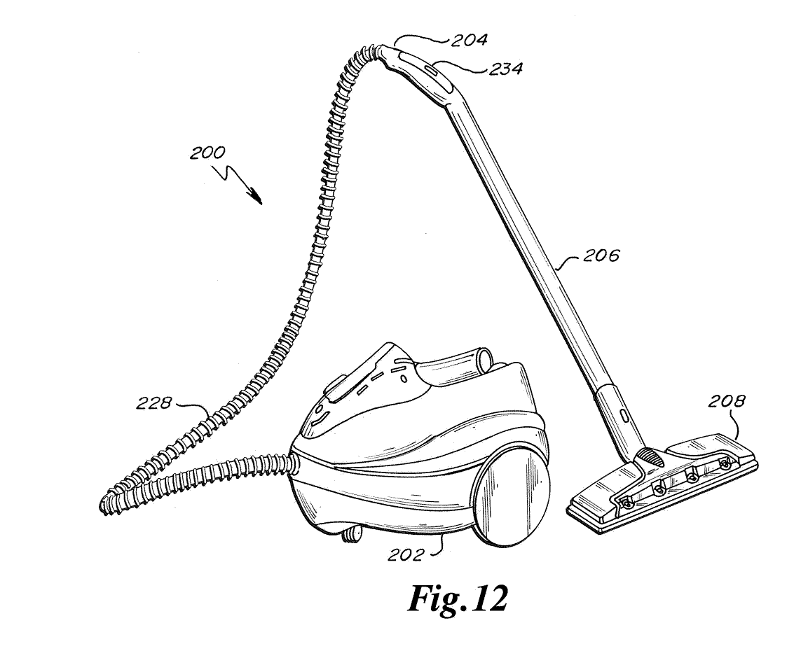

Reference is now made to FIGS. 12 through 22, where there is shown a steam cleaner/sanitizer 200 in the form of a canister steam generator 202 for heating water and having two reservoirs; a first reservoir for receiving the water to be heated into steam and a second reservoir for receiving a sanitizing agent. Flexible hose assembly 228 leads from the canister and includes two internal tubings; steam tube 120A connected to the steam reservoir and sanitizer tube 120B connected to the sanitizer reservoir. A floor cleaning accessory 104 includes a rigid hose assembly 106 and a steam-dispending head 108 connectable to the steam generator.

It should be understood that the disclosure constitutes merely a first exemplary one of the infinite number of possible embodiments for use in practicing the present invention. For instance, the steam generator 202 depicted is merely exemplary of typical such steam generators of the art and it should be understood that the invention may be practiced using another or most equivalent steam generators having means to accept water and a sanitizing agent, heat the water to steam for supplying there-from, and having means to supply such steam and the sanitizing agent to a dispensing means.

Water is added into an internal boiling chamber of the generator through a water-filling opening after its lid is temporarily removed. The lid is replaced after the boiling chamber is filled, and a heating element communicating with the boiling chamber is energized to heat the water within the chamber into steam, which is retained therein under pressure.

A sanitizing solution, preferably the solution later described or an equivalent thereto, is added into an internal sanitizer chamber of the generator through a sanitizer-filling opening after its lid is temporarily removed. The lid is replaced after the sanitizer chamber is filled, and the sanitizing agent is retained therein. A pump communicates with the sanitizer chamber to selectably force the sanitizer out through sanitizer tube 120B, when needed.

The rigid hose assembly 206 includes two parallel tubes 210F and 210B contained within a common rigid tubular housing 212, and a control valve 214. The control valve is actuated by a trigger 234 to force the valve into four distinct functions; “Off”, “Frontal Sanitizer/Bottom Steam”, “Frontal Steam/Bottom Sanitizer”, and “Frontal/Bottom Sanitizer/Steam”. Those functions and other alternate functions will be further explained.

The rigid hose assembly affixes in fluid communication to the flexible hose of the steam generator at the rigid hose's proximal end 230 such that the flexible hose's inlet tubes 120A and 120B connect to valve 214, and the rigid hose assembly is integrated with and in fluid communication with the steam-dispensing head 208 at the hose's distal end 232. The hose assembly thereby serves as a steam and sanitizer conduit from the steam generator 202 to the steam-dispensing head 208.

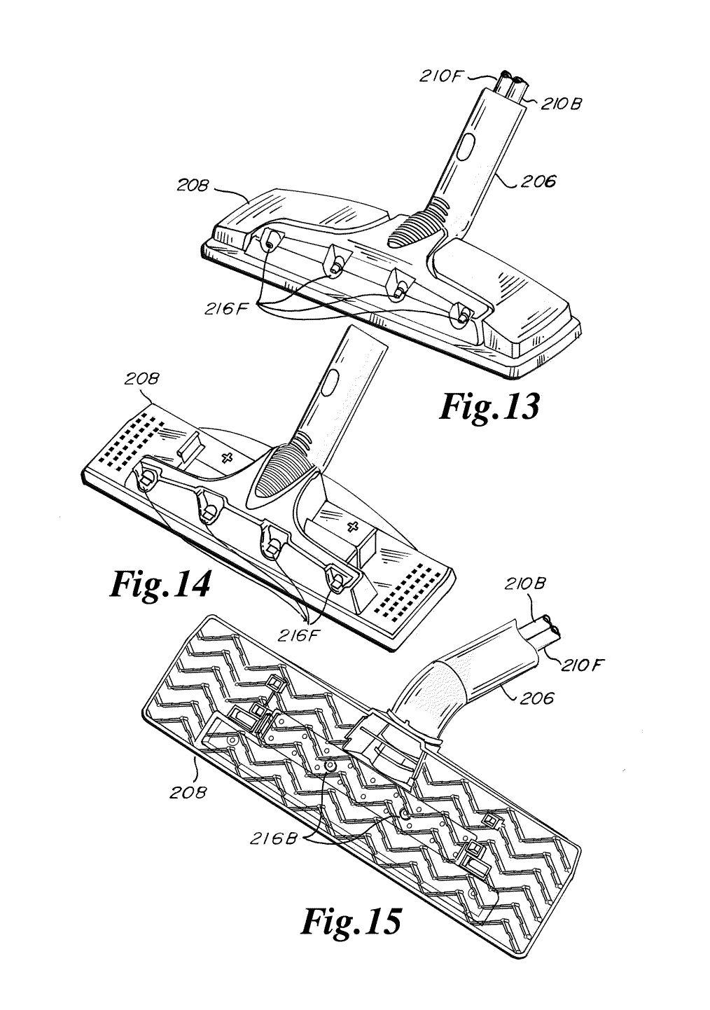

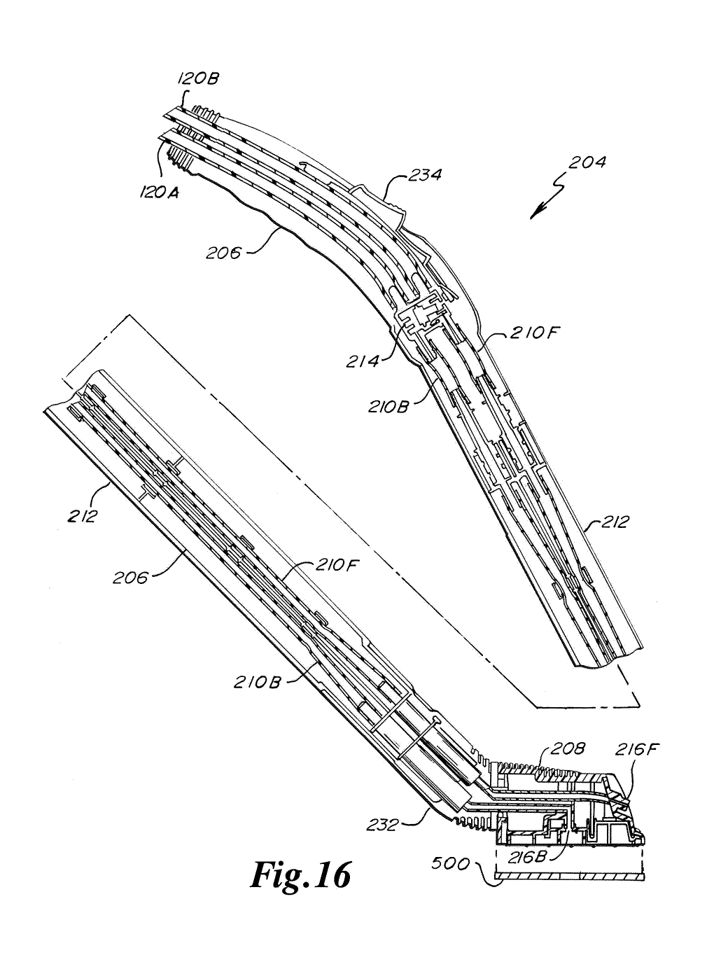

FIGS. 13 through 15 show that the dispensing head 208 is equipped with both frontal dispensing ports 216F, which are frontally-directed from the head, and bottom dispensing ports 216B, which are downwardly-directed from the head. FIG. 16 is a cross-section through the accessory 204 to show the fluid pathways there-through. The boiling and sanitizer chambers of the generator communicate with one or both of two tubes 210F and 210B of the hose assembly through inlet tubes 120A and 120B, respectively, and through the control valve 114, such that the valve normally denies escape of steam from the boiling chamber and denies escape of sanitizing agent from the sanitizer chamber, and such that activation of the valve into one of three positions allows the escape of steam and sanitizer from their respective chambers to and through the rigid hose to the steam-dispensing head. Preferably, the underside of the head has removably and replaceably attachable thereto pad 500, such as, but not limited to the type disclosed in priority Application No 13/013956. Preferably, the means for removably and replaceably attaching the pad to the underside of the head are “hook and loop” hooks on the underside.

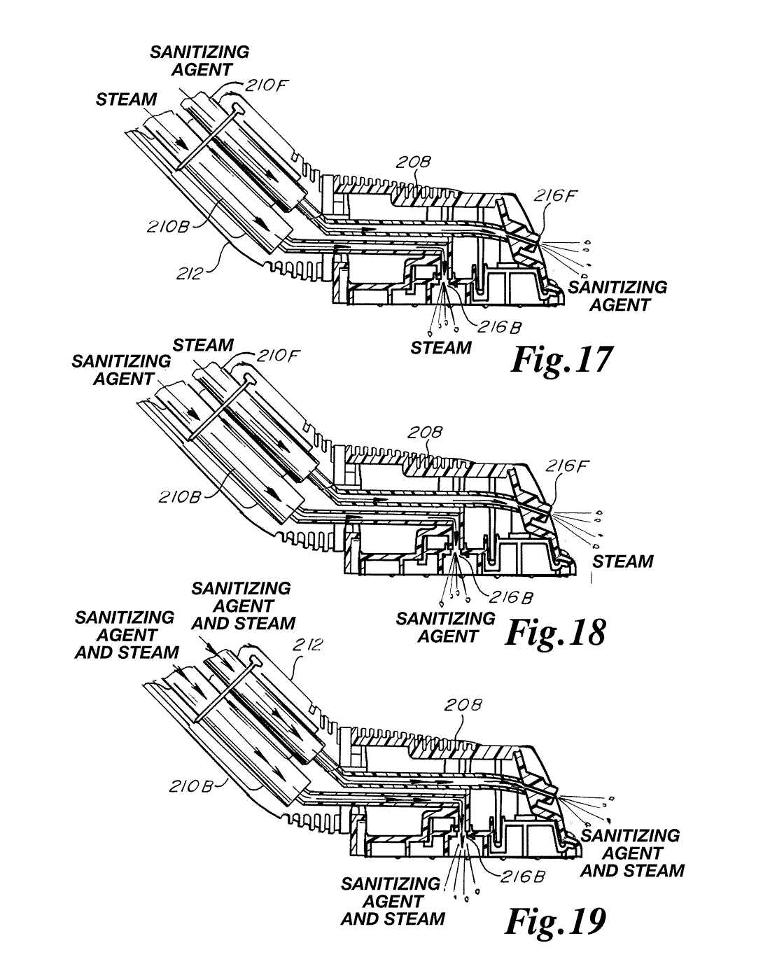

We refer now to FIGS. 17 through 19 and respectively corresponding FIGS. 20 through 22. These figures show the “Frontal Sanitizer/Bottom Steam”, “Frontal Steam/Bottom Sanitizer”, and “Frontal/Bottom Sanitizer/Steam” functions, respectively. The “Off” function is not shown, as in that position valve 214 is closed and denies steam and sanitizer to the dispensing head completely.

Sanitizer dispensing ahead of the dispensing head from frontal ports 216F in the “Frontal Sanitizer/Bottom Steam”, function, such as to sanitize the floor ahead of wiping the sanitizing agent up, is accomplished by the arrangement shown in FIGS. 17 and 20. Steam dispensing below the dispensing head from bottom ports 216B to penetrate into the floor below the dispenser as it is being wiped during this function is also shown in these figures. With dispensing head 208 placed upon and against the floor or surface and the trigger moved to force the control valve into the “Frontal Sanitizer/Bottom Steam” position shown in FIG. 20, sanitizing agent from the sanitizer chamber is allowed to flow through the valve to tube 210F and pressurized steam from the boiling chamber is allowed to flow through the valve to tube 210B. The sanitizing agent flows eventually and forcefully out through frontal ports 216F onto the floor or surface in front of the head as it is moved about the floor or surface. Pressurization from the pump of the generator causes the sanitizing agent to be forcefully squirted from the frontal ports. Steam flows eventually and forcefully out through bottom ports 216B onto the floor or surface below the head as it is moved about the floor or surface. Wiping pad 500 will then wipe up and absorb the sanitizer and the steam that condenses back to water as it contacts the floor.

Steam dispensing ahead of the dispensing head from frontal ports 216F in the “Frontal Steam/Bottom Sanitizer”, function, such as to soften residue and sterilize the floor ahead of wiping the dirty water up that results from condensation of the steam on the floor, is accomplished by the arrangement shown in FIGS. 18 and 21. Sanitizer dispensing below the dispensing head from bottom ports 216B to sanitize the floor below the dispenser as it is being wiped during this function is also shown in these figures. With dispensing head 208 placed upon and against the floor or surface and the trigger moved to force the control valve into the “Frontal Steam/Bottom Sanitizer” position shown in FIG. 21, pressurized steam from the boiling chamber is allowed to flow through the valve to tube 210F and sanitizer from the sanitizer chamber is allowed to flow through the valve to tube 210B. The steam flows eventually and forcefully out through frontal ports 216F onto the floor or surface in front of the head as it is moved about the floor or surface. Sanitizing agent flows eventually and forcefully out through bottom ports 216B onto the floor or surface below the head as it is moved about the floor or surface. Pressurization from the pump of the generator causes the sanitizing agent to be forcefully squirted from the bottom ports. Wiping pad 500 will then wipe up and absorb the sanitizer and the steam that condenses back to water as it contacts the floor.

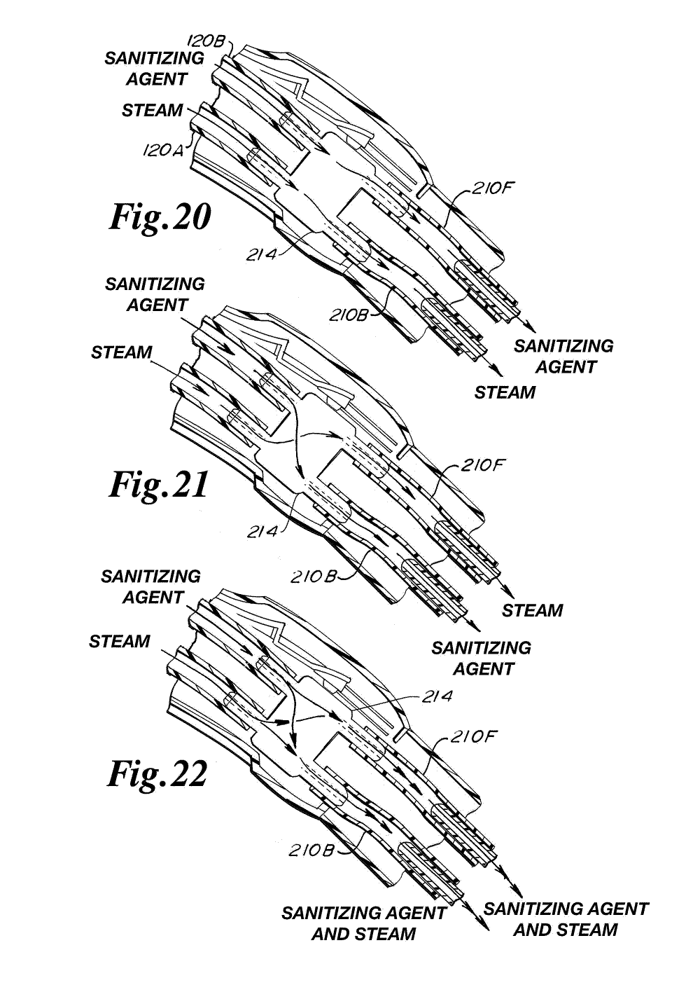

A mixture of steam and sanitizing agent is dispenses both ahead of and below the dispensing head in the “Frontal/Bottom Sanitizer/Steam” function accomplished by the arrangement shown in FIGS. 19 and 22. In this way, both of the aforesaid functions may be accomplished simultaneously, and the combination of steam and sanitizing agent it found to provide an unexpectedly superior result than treating the floor with steam and sanitizing agent separately.

During application of the mixture of steam and sanitizing agent of the Frontal/Bottom Sanitizer/Steam” function the valve is configured to control the relative amounts of the steam and sanitizing agent being dispensed at a volume rate of four parts sanitizing agent to ten parts water. The heating of this mixture is found to improve sanitization and sterilization both by an improvement in the function of the heated sanitizing agent over the same agent in a cooled state, and by an increased penetration of the sanitizing agent into the pores and fissures of the floor due to the penetrating effects of the steam.

Such a benefit is also realized, although to a lesser extent, by the sequential applications of steam and sanitizing agent as the dispensing head is moved back and forth over the floor during the “Frontal Sanitizer/Bottom Steam” and “Frontal Steam/Bottom Sanitizer” functions. These allow the sanitizing agent to contact the floor or surface ahead of the following steam, or vice-versa. In the “Frontal Sanitizer / Bottom Steam” function, the more concentrated sanitizing agent contacts the floor and is heated and forced into any porous features of the floor by the steam's penetrating qualities. This is found to improve sanitizing deeper into the floor and results in longer lasting of the sanitized conditions than separate application of steam and sanitizing agent. In the “Frontal Steam/Bottom Sanitizer” function, the more steam contacts and heats the floor ahead of the sanitizing solution and unexpectedly improves the sanitizing effects of the solution.

The preferred sanitizing agent is a liquid consisting of Active Ingredients in an inert base. The inert base is preferably water. The Active Ingredients preferably include:

0.125% Alkyl* dimethyl ethylbenzyl ammonium chloride *(68% C12, 32% C14);

0.125% BTC 2125, component of (with 069111); and

0.2% Chlorine dioxide.

Synonyms for and/or equivalents to Alkyl* dimethyl ethylbenzyl ammonium chloride *(68% C12, 32% C14) include:

BTC 2125M, component of (with 069104);

BX-673;

C12-14-alkyl(ethylbenzyl) dimethyl ammonium chlorides; and

Quaternary ammonium compounds, C12-14-alkyl((ethylphenyl)methyl)dimethyl, chlorides.

Synonyms for BTC 2125, component of (with 069111) include:

Alkyl* dimethyl benzyl ammonium chloride *(60% C14, 30% C16, 5% C18, 5% C12);

BTC 2125M (Use 2 code nos. 069104 and 069154);

BTC 776; and

BTC824.

Synonyms for and/or equivalents to Chlorine Dioxide include:

Chlorine Oxide; and

Chlorine Peroxide.

Through testing, it is found that the steam cleaning procedure described above and using the described mixture of steam and sanitizing agent kills 99.99% of at least Animal Pathogenic Bacteria (g− And G+ Vegetative), Aspergillus Niger, Mold, Mildew, Salmonella Enterica, Staphylococcus Aureus, E. Coli, Legionella Pneumophila, and Norovirus. Because killing is achieved by oxidation, it does not mask, but instead eliminates odors caused by such organisms. Other organic odors caused by such sources as fire and smoke are also eliminated. Through a combination of sterilization and sanitizing residue, microbial growth and odors are eliminated for extended periods over other forms of cleaning, in most cases up to seven days.

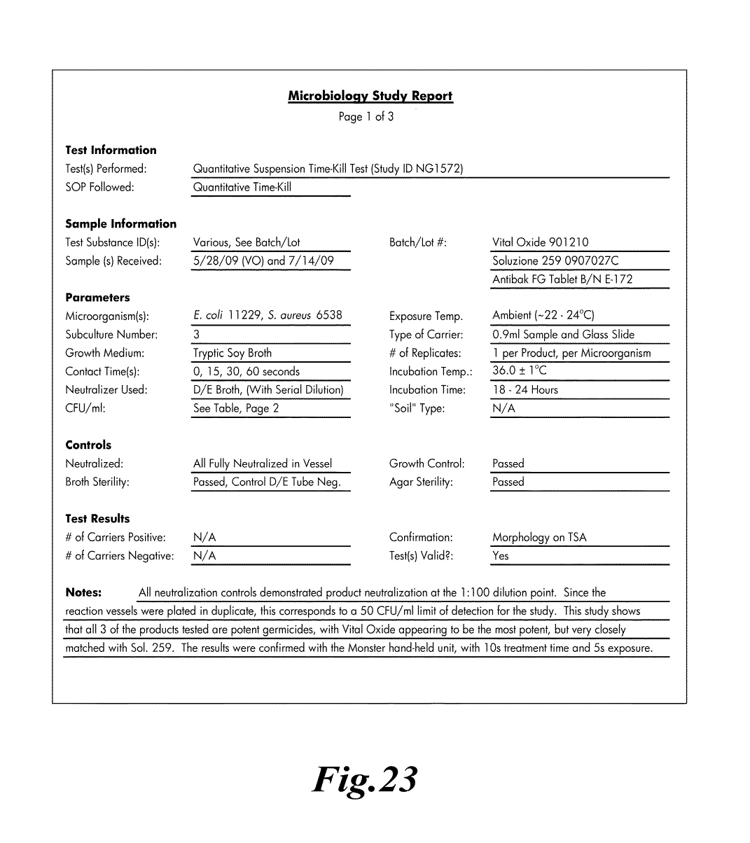

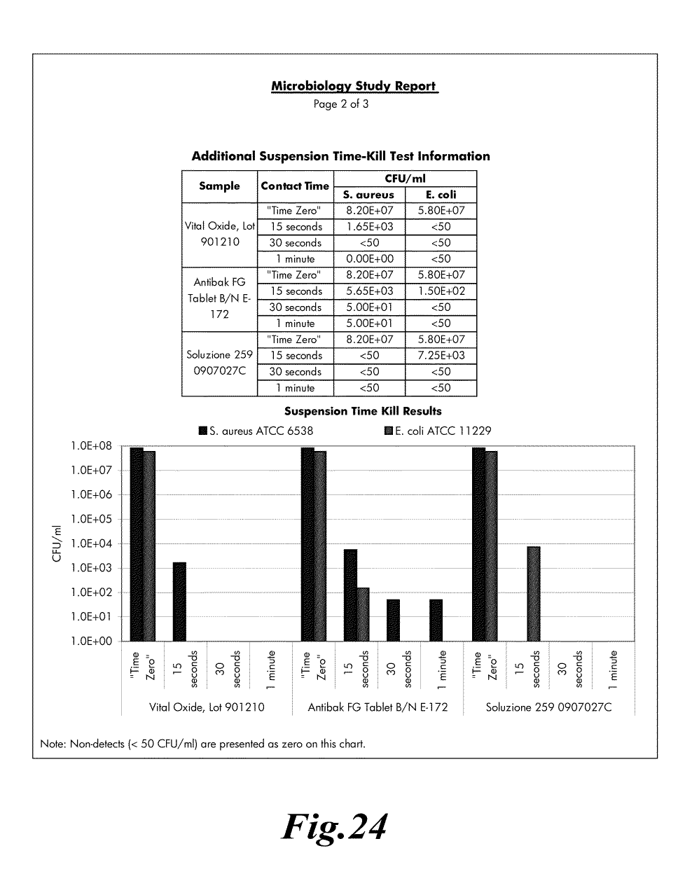

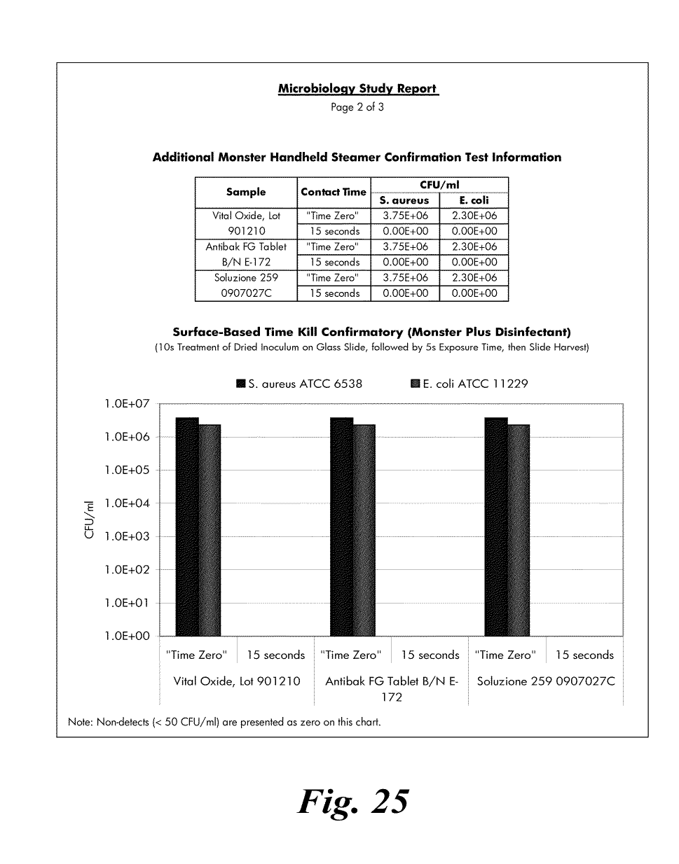

FIGS. 23 to 25 show a microbiology study report in which the apparatus of FIG. 12 was used as described with three alternative oxidizing agents;

Vital Oxide, (Chlorine dioxide 0.20%; Alkyl*dimethyl benzyl ammonium chloride*(60% C14, 30% C16, 5% C12, 5% C18) 0.125%, and Alkyl*dimethyl ethylbenzyl ammonium chloride*(68% C12, 32% C14) 0.125%);

Antibak FG (Potassium peroxymonosulphate 30-50%; Sodium dodecylbenzene sulphonate 0-5%; and Tetrasodium ethylene diamine tetraacetate 0-5%); and

Soluzione 259, a proprietary blend of the chemical components included above.

As shown in the report, rapid and thorough sanitization is achieved.

For instance, the arrangement could be employed in a steam pressing iron to better sanitize clothing during ironing. In such an arrangement, an iron with a water reservoir is provided with an extra reservoir for the afore-describe sanitizing agent, and a pair of triggers on the iron or a trigger-controlled valve in the iron could be selectively activated to expel either only steam when the “steam” trigger is pressed as in traditional steam ironing, or to expel both steam and sanitizing agent, either in a mixed form or from frontally-directed and downwardly-directed ports.

As indicated above, this invention may also be employed as an improvement to an upright steaming mop, such as that disclosed in related U.S. application Ser. No. 11/885994, the specification of which is incorporated herein by reference, in its entirety. In such, the sanitizing agent is stored in and dispensed from the “detergent” reservoir and is selectively dispensed onto the floor alone or in combination with the steam.

While the invention has been shown and described with reference to a specific exemplary embodiment, it should be understood by those skilled in the art that various changes in form and detail may be made without departing from the spirit and scope of the invention, and that the invention should therefore only be limited according to the following claims, including all equivalent interpretation to which they are entitled.

We claim:

1. An accessory for a steam generating appliance comprising:
a valve having one or more inlets for receiving one or both of steam and a sanitizing agent from the steam generating appliance;
one or more forwardly-directed outlet ports and one or more downwardly-directed outlet ports;
a first conduit providing fluid communication between the valve and the one or more forwardly-directed outlet ports;
a second conduit providing fluid communication between the valve and the one or more downwardly-directed outlet ports;
a valve controller for selectively allowing or denying the one or both of steam and a sanitizing agent to flow through the valve and conduits to the outlet ports in at least the following modes;
denying the one or both of steam and a sanitizing agent to flow to and from either of the forwardly-directed or the downwardly-directed outlet ports;
allowing the one or both of steam and a sanitizing agent to flow to and from both of the forwardly-directed and the downwardly-directed outlet ports;
allowing the one or both of steam and a sanitizing agent to flow to and from only the one or more forwardly-directed outlet ports; and
allowing the one or both of steam and a sanitizing agent to flow to and from only the one or more downwardly-directed outlet ports.

2. The accessory of claim 1 further comprising a handle, a rigid tubular wand, and a dispensing head, wherein;
the valve is disposed in the handle;
the first and second conduits are disposed in the wand;
and the outlet ports are disposed in the dispensing head.

3. The accessory of claim 2 wherein the one or more downwardly-directed ports are disposed in a bottom surface of the dispensing head; and the bottom surface comprises means for removably receiving an absorbent pad.

4. The accessory of claim 3 wherein the means for removably receiving the absorbent pad comprises “hook and loop” hooks.

5. The accessory of claim 4 wherein the dispensing head comprises the received absorbent pad, and the pad comprises a super-absorbent material.

6. The accessory of claim 5 wherein the super-absorbent material comprises hydrophilic fibers and hydrogel-forming particles.

7. The accessory of claim 5 wherein the super-absorbent material comprises cotton and sodium polyacrylate.

9. An accessory for a steam generating appliance comprising:
a valve having an inlet for receiving steam from the steam generating appliance;
one or more forwardly-directed outlet ports and one or more downwardly-directed outlet ports;
a first conduit providing fluid communication between the valve and the one or more forwardly-directed outlet ports;
a second conduit providing fluid communication between the valve and the one or more downwardly-directed outlet ports;
a valve controller for selectively allowing or denying the steam to flow through the valve and conduits to the outlet ports in at least the following modes;
denying the steam to flow to and from either of the forwardly-directed or the downwardly-directed outlet ports;
allowing the steam to flow to and from both of the forwardly-directed and the downwardly-directed outlet ports;
allowing the steam to flow to and from only the one or more forwardly-directed outlet ports; and
allowing the steam to flow to and from only the one or more downwardly-directed outlet ports.

10. The accessory of claim 9 further comprising a dispensing portion and an absorbent pad;
wherein the one or more downwardly-directed ports are disposed in a bottom surface of the dispensing portion; and
the bottom surface comprises means for removably receiving the absorbent pad.

11. The accessory of claim 10 wherein the absorbent pad comprises an opening there-through shaped and disposed to avoid the one or more downwardly-directed ports, such that steam flowing there-from does not impact the pad.

12. The accessory of claim 11 wherein the pad comprises a super-absorbent material.

13. The accessory of claim 12 wherein the super-absorbent material comprises hydrophilic fibers and hydrogel-forming particles.

14. The accessory of claim 12 wherein the super-absorbent material comprises cotton and sodium polyacrylate.

15. The accessory of claim 9 further comprising a handle, a rigid tubular wand, and a dispensing portion, wherein;
the valve is disposed in the handle;
the first and second conduits are disposed in the wand;
and the outlet ports are disposed in the dispensing portion.

16. The accessory of claim 15 wherein the one or more downwardly-directed ports are disposed in a bottom surface of the dispensing portion; and the bottom surface comprises means for removably receiving an absorbent pad.

17. An accessory for a steam generating appliance comprising:
a valve having a first inlet for receiving steam from the steam generating appliance and a second inlet for receiving a sanitizing agent;
one or more forwardly-directed outlet ports and one or more downwardly-directed outlet ports;
a first conduit providing fluid communication between the valve and the one or more forwardly-directed outlet ports;
a second conduit providing fluid communication between the valve and the one or more downwardly-directed outlet ports;
a valve controller for selectively allowing or denying the steam and the sanitizing agent to flow through the valve and conduits to the outlet ports in at least the following modes;
denying both the steam and the sanitizing agent to flow to and from either of the forwardly-directed or the downwardly-directed outlet ports;
allowing the steam to flow to and from the one or more forwardly-directed outlet ports and the sanitizing agent to flow to and from the one or more downwardly-directed outlet ports;
allowing the sanitizing agent to flow to and from the one or more forwardly-directed outlet ports and the steam to flow to and from the one or more downwardly-directed outlet ports; and
allowing a mixture of the steam and the sanitizing agent to flow to and from both of the forwardly-directed and the downwardly-directed outlet ports.

18. The accessory of claim 17 in combination with the sanitizing agent, wherein the sanitizing agent comprises Alkyl Dimethyl Ethylbenzyl Ammonium Chloride.

19. The combination of claim 18 wherein the percentage of Alkyl Dimethyl Ethylbenzyl Ammonium Chloride by volume of the sanitizing agent is approximately 0.125.

20. The accessory of claim 17 in combination with the sanitizing agent, wherein the sanitizing agent comprises BTC 2125.

21. The combination of claim 20 wherein the percentage of BTC 2125 by volume of the sanitizing agent is approximately 0.125.

22. The accessory of claim 17 in combination with the sanitizing agent, wherein the sanitizing agent comprises Chlorine Dioxide.

23. The combination of claim 22 wherein the percentage of Chlorine Dioxide by volume of the sanitizing agent is approximately 0.20.

24. The accessory of claim 17 in combination with the sanitizing agent, wherein the sanitizing agent comprises Alkyl Dimethyl Ethylbenzyl Ammonium Chloride, BTC 2125, and Chlorine Dioxide.

25. The combination of claim 24 wherein the percentage of Alkyl Dimethyl Ethylbenzyl Ammonium Chloride by volume of the sanitizing agent is approximately 0.125.

26. The accessory of claim 17 further comprising a handle, a rigid tubular wand, and a dispensing head, wherein;
the valve is disposed in the handle;
the first and second conduits are disposed in the wand;
and the outlet ports are disposed in the dispensing head.

27. The accessory of claim 26 wherein the one or more downwardly-directed ports are disposed in a bottom surface of the dispensing head; and the bottom surface comprises means for removably receiving an absorbent pad.
